(19) United States
(12) Patent Application Publication
(10) Pub. No.: US 2004/0211583 A1
(43) Pub. Date: Oct. 28, 2004

(54) Surface-mount-type high-frequency module

(76) Inventor: Masaru Kosaka, Osaka-shi (JP)

Correspondence Address: BIRCH STEWART KOLASCH & BIRCH, PO BOX 747, FALLS CHURCH, VA, 22040-0747, US

(21) Appl. No.: 10/819,937

(22) Filed: Apr. 8, 2004

(30) Foreign Application Priority Data
Apr. 23, 2003 (JP) 2003-118773

(51) Int. Cl. H05K 5/00; A61K 31/35; H02G 3/08; A01N 43/16

(52) U.S. Cl. 174/52.1

(57) ABSTRACT

A surface-mount-type high-frequency module in accordance with one or more embodiments of the present invention comprises BGA package IC(s) 1 and printed circuit board(s) carrying such BGA package IC(s) 1; such printed circuit board(s) being multilayer board(s), laminated at which there are inner layer(s) comprising core material(s) 6, and outer layer(s) constituting exterior(s) relative to inner layer(s) and comprising build-up layer(s) 5. Top surface(s) of printed circuit board(s) (core material(s) 6), in region(s) where BGA package IC(s) 1 carried thereby is/are mounted, is/are formed so as to be lower than top surface(s) of outer layer(s) (build-up layer(s) 5). Gap(s) between core material(s) 6 and BGA package IC(s) 1 is/are filled with encapsulant resin(s) 3.

### CROSS-REFERENCE TO RELATED APPLICATION/PRIORITY

This Nonprovisional application claims priority under 35 U.S.C. §119(a) on Patent Application No. 2003-118773 filed in Japan on Apr. 23, 2003, the entire contents of which are hereby incorporated by reference.

### TECHNICAL FIELD

The present invention pertains to a surface-mount-type high-frequency module having printed circuit board(s) on which BGA (Ball Grid Array) package IC(s) is/are mounted.

### BACKGROUND ART

There has heretofore been an ever-increasing need for miniaturization (i.e., low-profile and lightweight design) of mobile telephones, PDAs, laptop-type personal computers, and other such electronic equipment containing high-frequency modules and employed as wireless communication devices. And in recent years, it is primarily modules which permit surface mounting that have come to be the favored approach as the high-frequency modules which are employed in such electronic equipment.

FIG. 2 is an oblique view showing a conventional surface-mount-type high-frequency module. Mounted on such a surface-mount-type high-frequency module there will sometimes be a shield cover (not shown) for controlling unwanted electromagnetic radiation emanating from the surface-mount-type high-frequency module body.

As means for accomplishing surface mounting of the surface-mount-type high-frequency module, terminal regions for input/output of electrical signals to/from the surface-mount-type high-frequency module take the form of edge through-holes 22 at the side surfaces of the printed circuit board 21 which forms the surface-mount-type high-frequency module, and all of the chip resistors, chip capacitors, chip inductors, laminated filters, and other such electronic components 23 making up the surface-mount-type high-frequency module are mounted on the surface at one side of printed circuit board 21; furthermore, no electronic components 23 being mounted on the surface at the other side thereof, by installing this surface of printed circuit board 21 on which no electronic components 23 are mounted such that it faces a board at the electronic equipment body and soldering this thereto it is possible to surface mount same to the electronic equipment body.

Now, where wireless communication is carried out between two or more pieces of electronic equipment, baseband signals capable of being processed by a microcomputer are converted into high-frequency signals as communication is being carried out. Indispensable to a high-frequency module for which ability to carry out modulation and demodulation between this baseband signal and this high-frequency signal is indispensable is that it be equipped with a transceiver IC 24 having modulation and demodulation capability. It will be necessary that such a high-frequency module be small and have a low profile. For this reason, ICs of the configuration referred to as BGA packages have come to be widely used as packages suitable for high-density mounting of transceiver IC 24 thereto. The I/O terminals for this BGA package IC are created by forming solder bumps at the IC backside.

FIG. 3 is a sectional view showing a location at which BGA package IC 32 is mounted to printed circuit board 31 of a surface-mount-type high-frequency module.

Solder land 34 comprising conductor formed on printed circuit board 31 is soldered to solder bump 35 formed on BGA package IC 32 so as to mount BGA package IC 32 to printed circuit board 31 and electrically connect this BGA package IC 32 and this printed circuit board 31.

However, when BGA package IC 32 is used, there are the following problems. There is a difference in the coefficients of thermal expansion at substrate 33 employed within BGA package IC 32 and printed circuit board 31 employed at the high-frequency module, as a result of which the degree to which shrinkage occurs at the respective materials in accompaniment to changes in temperature will be different between substrate 33 of BGA package IC 32 and printed circuit board 31 of the high-frequency module.

As a result, forces act where they are not needed, creating stresses, at solder joints between solder bumps 35 of BGA package IC 32 and the board/substrate to either side thereof. This can cause cracks to appear at the interfaces between solder bumps 35 and substrate 33 of BGA package IC 32 and/or at the solder joints between solder bumps 35 and printed circuit board 31 of the high-frequency module, resulting in open-type failures and causing the high-frequency module to no longer function electrically as such.

Furthermore, ICs are typically used in a wide variety of different types of equipment, and it is not assumed when they are being designed that they will be installed in a single type of equipment. And also with respect to printed circuit board 31 used in the module, the required materials will vary depending upon required thickness and laminate structure. This being the case, it is not practical to use the same material for substrate 33 employed at the IC and board 31 employed at the module.

As one method for dealing with such issues, a method has been provided as disclosed, for example, at Japanese Patent Application Publication Kokai No. 2002-33419 in which problems due to differences in coefficient of thermal expansion are avoided by using resin encapsulation to fill the gap between board 31 of the high-frequency module and BGA package IC 32.

However, where resin encapsulation is carried out as in such conventional art, problems such as the following still remain when same is applied to small surface-mount-type high-frequency modules.

FIG. 4 is a sectional view of a region at which a BGA package IC is mounted at a surface-mount-type high-frequency module in a situation where such resin encapsulation has been carried out.

Printed circuit boards used for surface-mount-type high-frequency modules typically employ a multilayer construction in order to permit high-density wiring. Often used as multilayer boards are build-up boards composed of core material 46, forming the inner layer portion thereof, and build-up layer(s) 45 forming outer layer(s) thereof.

Moreover, where resin encapsulation is used to fill the gap between such a build-up board and BGA package IC 41, the fact that encapsulant resin 43 is a substance possessing fluidity means that it is difficult to freely control the manner of its spreading, as a result of which it flows and spreads so as to occupy a broad band-like region surrounding BGA package IC 41.

For this reason, employment thereof with small surface-mount-type high-frequency modules can result in a situation in which, when BGA package IC 41 is near edge through-holes 44 formed as I/O terminal electrodes, encapsulant resin 43 flows into edge through-holes 44; and edge through-holes 44 being covered by encapsulant resin 43, they will no longer be usable as I/O terminal electrodes. It has therefore been the case with small surface-mount-type high-frequency modules of the type having edge through-holes 44 that resin encapsulation was impossible, making it necessary to depend on the reliability of the solder joint between solder bump 42 of BGA package IC 41 and land 47 of the printed circuit board.

Furthermore, where resin encapsulation has been possible it has been necessary to increase the distance L between BGA package IC 41 and edge through-holes 44 so as to prevent encapsulant resin 43 from flowing into edge through-holes 44, but this has represented an obstacle to achieving the small size which should be characteristic of high-frequency modules.

### SUMMARY OF INVENTION

In order to solve one or more of the aforementioned problems and/or other problems, a surface-mount-type high-frequency module in accordance with one or more embodiments of the present invention comprises one or more BGA package ICs; and one or more printed circuit boards carrying at least one of the BGA package IC or ICs; wherein at least one of the printed circuit board or boards is at least one multilayer board at which at least one inner layer and at least one outer layer constituting at least one exterior relative to at least one of the inner layer or layers are laminated; at least one top surface, in at least one region where at least one of the BGA package IC or ICs carried thereby is mounted, of at least one of the printed circuit board or boards is formed so as to be lower than at least one top surface of at least one of the outer layer or layers; and at least one gap between at least one of the printed circuit board or boards and at least one of the BGA package IC or ICs mounted to at least one of the printed circuit board or boards is filled with at least one resin. In accordance with such a constitution, encapsulant resin(s) may be impeded by wall(s) at region(s) peripheral to substrate location(s) at which BGA package IC(s) is/are mounted, permitting resin encapsulation to proceed without superfluous flowing thereof.

Furthermore, it is preferred that the at least one top surface in at least one mounting region of at least one of the printed circuit board or boards be of such structure as to cause it to be sufficiently lower than the at least one top surface of at least one of the outer layer or layers to prevent the at least one resin with which filling is carried out from flowing outward beyond at least one desired extent from at least one periphery of at least one of the BGA package IC or ICs. This will make it possible to prevent flowing of encapsulant resin(s) into superfluous region(s), making it possible to prevent encapsulant resin(s) from flowing into edge through-hole(s).

Moreover, at least one of the outer layer or layers may be formed so as to more or less completely surround at least one of the BGA package IC or ICs. In accordance with such a constitution, it will be possible to cause unwanted stress(es) acting at solder joint region(s) between BGA package IC(s) and printed circuit board(s) to be distributed so as to also act at region(s) other than solder joint region(s).

At least one of the outer layer or layers may be constituted so as to comprise at least one build-up layer, in which case same may be processed separately from inner layer substrate(s).

At least one edge through-hole may be disposed at at least one peripheral rim region of at least one of the printed circuit board or boards, in which case it will be possible for surface mounting of high-frequency module(s) to electronic equipment body or bodies to be easily carried out.

Surface-mount-type high-frequency modules in accordance with one or more embodiments of the present invention may thus prevent spreading of encapsulant resin(s) beyond desired extent(s) to region(s) where same is unwanted; and may, even with small surface-mount-type high-frequency modules, inhibit flowing of resin(s) into edge through-hole(s) serving as electrode terminal(s). As a result of being made to have such edge through-hole(s) and as a result of filling of space(s) between BGA package IC(s) and printed circuit board(s) with resin(s), it is possible to achieve manufacture of a small surface-mount-type high-frequency module with excellent reliability with respect to soldering between BGA package IC(s) and printed circuit board(s).

### BRIEF DESCRIPTION OF DRAWINGS

FIG. 1 is a sectional view of a region at which an IC is mounted at a surface-mount-type high-frequency module in accordance with the present invention.

FIG. 2 is an oblique view of a surface-mount-type high-frequency module in accordance with the conventional art.

FIG. 3 is a sectional view of a region at which an IC is mounted in accordance with the conventional art.

FIG. 4 is a sectional view of a region at which an IC is mounted at a surface-mount-type high-frequency module in accordance with the conventional art.

### DESCRIPTION OF PREFERRED EMBODIMENTS

Below, embodiments of the present invention are described with reference to the drawings.

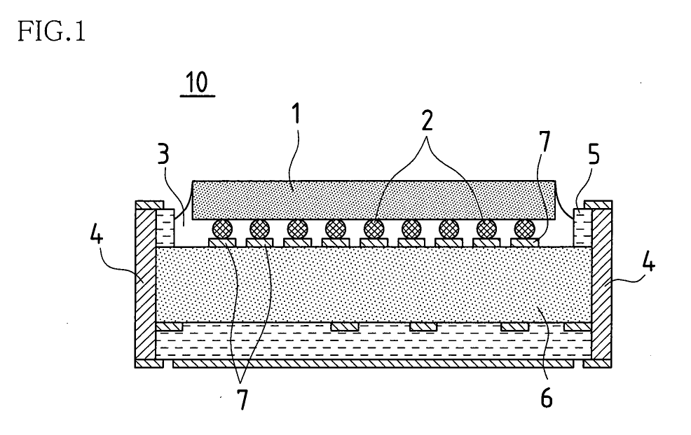

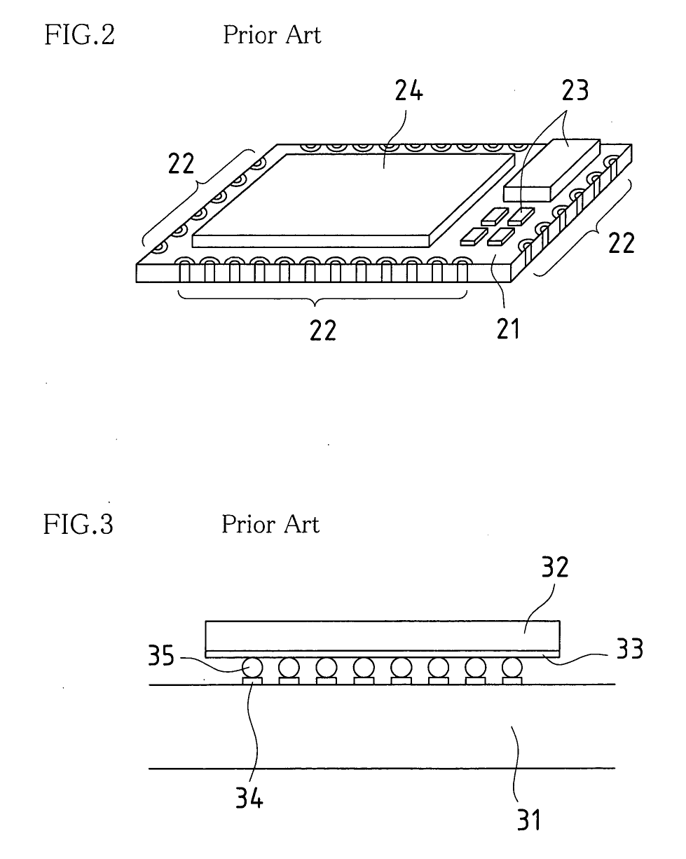

FIG. 1 is a sectional view showing surface-mount-type high-frequency module 10 in accordance with the present invention.

As shown in FIG. 1, surface-mount-type high-frequency module 10 of the present embodiment comprises BGA package IC 1 and a printed circuit board carrying this BGA package IC 1; this printed circuit board being a multilayer board, laminated at which there are an inner layer comprising core material 6, and an outer layer constituting an exterior relative to the inner layer and comprising build-up layer 5. Furthermore, the top surface of the printed circuit board (core material 6), in the region where BGA package IC 1 which is carried thereby is mounted, is formed so as to be lower than the top surface of the outer layer (build-up layer 5).

Furthermore, provided at the top surface of core material 6 there are solder lands 7 for making electrical connection between an electric circuit formed at core material 6 and solder bumps 2 formed at BGA package IC 1. These solder bumps 2 and these solder lands 7 are soldered together so as to cause BGA package IC 1 and the core material to be electrically connected.

Moreover, the gap between core material 6 and BGA package IC 1 is filled with encapsulant resin 3.

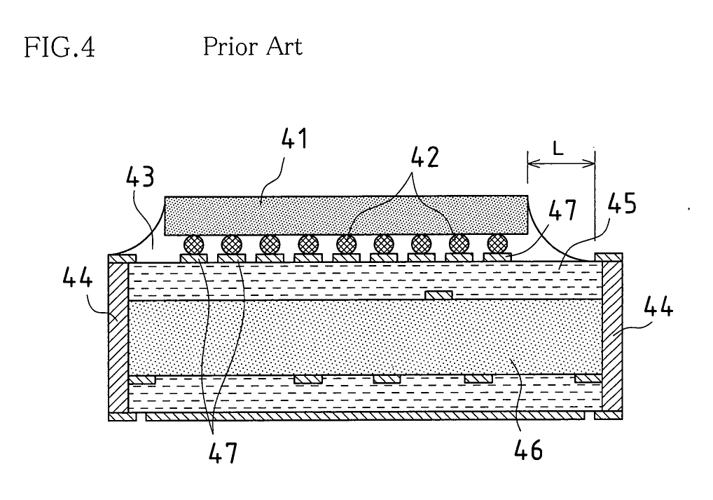

As with the conventional art shown in FIG. 4, this surface-mount-type high-frequency module 10 is such that unwanted portions of the build-up layer at locations corresponding to where BGA package IC 1 is carried thereby are removed by etch processing and/or laser processing so as to form build-up layer 5 as an outer layer over the core material.

Here, processing is carried out so as to cause the height of build-up layer 5 serving as wall for stopping flow of encapsulant resin to be such that the backside of BGA package IC 1 lies below this wall.

Next, BGA package IC 1 is installed in the concavity obtained as a result of using the foregoing processing to remove unwanted portions of the build-up layer at locations other than where build-up layer 5 will be used as a wall for stopping flow of encapsulant resin as described above, and solder lands 7 formed at the top of core material 6 and solder bumps 2 are soldered and joined so as to electrically connect BGA package IC 1 and core material 6.

A desired amount of encapsulant resin 3 such as will not cause the height of build-up layer 5, serving as wall for stopping flow of encapsulant resin, to be surmounted is then used to fill the gap between BGA package IC 1 and core material 6, and is cured.

As a result of adoption of such a surface-mount-type high-frequency module structure, it is possible to cause encapsulant resin to be impeded by the wall of build-up layer 5, permitting resin encapsulation to proceed without superfluous flowing thereof and permitting provision of a surface-mount-type high-frequency module having improved reliability with respect to soldering of BGA package IC 1. Furthermore, because encapsulant resin does not flow into edge through-holes 4, BGA package-type IC(s) can be mounted near edge through-hole(s) 4; and, there being no need to make provision for some distance(s) between edge through-hole(s) 4 and IC(s), high-density mounting is permitted and the surface-mount-type high-frequency module can be made smaller in size.

Moreover, if build-up layer 5, constituting the outer layer, is formed so as to more or less completely surround BGA package IC 1, it will be possible to fill the region between the printed circuit board and BGA package IC 1 with encapsulant resin such that there is no gap therebetween, and this will make it possible for unwanted stresses acting at solder joint region(s) between BGA package IC 1 and the printed circuit board to be distributed so as to also act at region(s) other than solder joint region(s), as a result of which it will be possible to reduce the likelihood of separation at solder joint region(s) due to cracking.

Furthermore, where the outer layer is a build-up layer 5 formed in laminated fashion with respect to the inner layer substrate, it will be possible to process same separately from the inner layer substrate, facilitating fabrication of wall(s) at location(s) peripheral to region(s) at which BGA package IC(s) 1 is/are mounted.

If edge through-hole(s) 4 is/are provided at peripheral rim region(s) of printed circuit board(s), it will be possible to easily carry out surface mounting of high-frequency module(s) to electronic equipment body or bodies; and moreover, it will be possible to easily ascertain condition(s) of solder joint(s) between printed circuit board(s) of electronic equipment body or bodies and high-frequency module(s).

The present invention may be embodied in a wide variety of forms other than those presented herein without departing from the spirit or essential characteristics thereof. The foregoing embodiments and working examples, therefore, are in all respects merely illustrative and are not to be construed in limiting fashion. The scope of the present invention being as indicated by the claims, it is not to be constrained in any way whatsoever by the body of the specification. All modifications and changes within the range of equivalents of the claims are moreover within the scope of the present invention.

1. A surface-mount-type high-frequency module comprising:
one or more BGA package ICs; and
one or more printed circuit boards carrying at least one of the BGA package IC or ICs;
wherein at least one of the printed circuit board or boards is at least one multilayer board at which at least one inner layer and at least one outer layer constituting at least one exterior relative to at least one of the inner layer or layers are laminated;
at least one top surface, in at least one region where at least one of the BGA package IC or ICs carried thereby is mounted, of at least one of the printed circuit board or boards is formed so as to be lower than at least one top surface of at least one of the outer layer or layers; and
at least one gap between at least one of the printed circuit board or boards and at least one of the BGA package IC or ICs mounted to at least one of the printed circuit board or boards is filled with at least one resin.

2. A surface-mount-type high-frequency module according to claim 1 wherein:
the at least one top surface in at least one mounting region of at least one of the printed circuit board or boards is of such structure as to cause it to be sufficiently lower than the at least one top surface of at least one of the outer layer or layers to prevent the at least one resin with which filling is carried out from flowing outward beyond at least one desired extent from at least one periphery of at least one of the BGA package IC or ICs.

3. A surface-mount-type high-frequency module according to claim 1 wherein:
at least one of the outer layer or layers is formed so as to more or less completely surround at least one of the BGA package IC or ICs.

4. A surface-mount-type high-frequency module according to claim 1 wherein:
at least one of the outer layer or layers comprises at least one build-up layer.

5. A surface-mount-type high-frequency module according to claim 3 wherein:
at least one of the outer layer or layers comprises at least one build-up layer.

6. A surface-mount-type high-frequency module according to claim 1 wherein:
at least one edge through-hole is disposed at at least one peripheral rim region of at least one of the printed circuit board or boards.

7. A surface-mount-type high-frequency module according to claim 3 wherein:
at least one edge through-hole is disposed at at least one peripheral rim region of at least one of the printed circuit board or boards.

8. A surface-mount-type high-frequency module according to claim 4 wherein:
at least one edge through-hole is disposed at at least one peripheral rim region of at least one of the printed circuit board or boards.

9. A surface-mount-type high-frequency module according to claim 5 wherein:
at least one edge through-hole is disposed at at least one peripheral rim region of at least one of the printed circuit board or boards.

10. A surface-mount-type high-frequency module according to claim 2 wherein:
at least one of the outer layer or layers is formed so as to more or less completely surround at least one of the BGA package IC or ICs.

11. A surface-mount-type high-frequency module according to claim 2 wherein:
at least one of the outer layer or layers comprises at least one build-up layer.

12. A surface-mount-type high-frequency module according to claim 2 wherein:
at least one edge through-hole is disposed at at least one peripheral rim region of at least one of the printed circuit board or boards.

13. A surface-mount high-frequency module comprising:
a circuit board comprising a top surface having a depression therein;
a BGA package IC mounted in said depression; and
a resin at least partially surrounding said BGA package IC and substantially filling said depression.

14. A surface-mount high-frequency module according to claim 10 wherein said circuit board comprises a multi-layer printed circuit board comprising an inner layer having a top surface and an outer layer having a top surface, said depression being formed in said outer layer.

15. A surface-mount high-frequency module according to claim 11 wherein said BGA package is mounted in said depression and connected to said inner layer outer surface.
